(19) United States
(12) Patent Application Publication
(10) Pub. No.: US 2021/0085743 A1
(43) Pub. Date: Mar. 25, 2021

(54) SARRACENIA-BASED TOPICAL COMPOSITIONS AND METHODS OF USE THEREOF

(71) Applicant: Aubio Life Sciences, LLC, Wixom, MI (US)

(72) Inventors: Kim SHAFER, White Lake, MI (US); Yvan ROCHON, Sedona, AZ (US)

(73) Assignee: Aubio Life Sciences, LLC, Wixom, MI (US)

(21) Appl. No.: 16/580,812

(22) Filed: Sep. 24, 2019

(51) Int. Cl. A61K 36/886; A61K 36/53; A61K 36/484; A61K 36/38; A61K 36/82; A61K 36/254; A61K 31/4166; A61K 31/167; A61K 31/045; A61K 9/00; A61P 17/02

(52) U.S. Cl. CPC A61K 36/886; A61K 36/53; A61K 36/484; A61K 36/38; A61K 36/82; A61P 17/02; A61K 31/4166; A61K 31/167; A61K 31/045; A61K 9/0014; A61K 36/254

(57) ABSTRACT

Methods, compositions, and protocols are disclosed comprising topical therapies for viral infections and for immune modulation in diseases including skin disorders and disorders that cause skin manifestations. Specifically, the invention pertains to formulations comprising Sarracenia extracts, and methods of use thereof.

### Field of the Invention

The invention pertains to topical therapies for viral infections and for immune modulation in diseases including skin disorders and disorders that cause skin manifestations. Specifically, the invention pertains to formulations comprising extracts of pitcher plants, preferably Sarracenia extracts, and methods of use thereof.

### BACKGROUND OF THE INVENTION

Pitcher plants comprise several species of carnivorous plants that have specialized leaves capable of trapping prey. These traps attract insects using visual lures (e.g. color of anthocyanin pigment) and nectar, and the prey then becomes entrapped and drowned in the nectar and digested. Pitcher plants include members of the Nepenthaceae and Sarraceniaceae families, the two most common families, as well as Cephalotaceae and Bromeliaceae. In addition to pitcher plants, other families of carnivorous plants exert similar functions; e.g. Venus flytrap (Dionaea muscipula) and sundews (Drosera).

Sarraceniaceae is a New World carnivorous plant family comprising three genera: Darlingtonia, Heliamphora, and Sarracenia. Sarracenia is a genus of New World carnivorous plants that grow in nutrient deficient wetland environments. Many species and subspecies that have been identified: Sarracenia leucophylla, Sarracenia alata, Sarracenia flava, Sarracenia jonesii, Sarrcenia minor, Sarracenia oreophila, Sarracenia psittacina, Sarracenia purpurea, Sarracenia rosea, and Sarracenia rubra. In general, the Sarraceniaceae family is poorly characterized in terms of its chemical composition, however, it has been determined that its members have some compounds in common and others that are unique for individual variants [1].

The purple pitcher plant, Sarracenia purpurea L., is widely distributed in North America and has a history of use in traditional medicine. The plant has long been recognized among aboriginal peoples in Canada as having medicinal properties; specifically, for treatment of the symptoms of type 2 diabetes and to improve wound healing [2]. This species of Sarracenia has been most extensively studies for the anti-viral properties of its extracts. Studies have investigated the cytoprotective actions of leaf and root extracts of Sarracenia purpurea L in in vitro models where cells are exposed to high glucose concentrations to mimic the conditions of diabetes. The results showed that leaf extracts reduced glucose-induced cell loss in a concentration-dependent manner [3]. Another study indicated that Sarracenia leaf extract contains compounds that stimulate glucose uptake by muscle cells [4]. Information related to how Sarracenia extracts protect cells from cytotoxicity, and their broader effects on cellular metabolism and functions, remains to be elucidated.

Extracts of Sarracenia purpurea are inhibitors of poxvirus replication at the level of early viral transcription [5]. This activity toward Orthopoxviruses is consistent with the historical reports of S. purpurea as a therapy for smallpox. Sarracenia extracts have also been used in traditional medicine for treating the symptoms of tuberculosis. An analysis of methanolic extracts of Sarracenia purpurea showed that betulinaldehyde, betulinic acid, and ursolic acid are the principal compounds that are responsible for the antimycobacterial activity of S. purpurea [6]. Activity of components of Sarracenia against herpes simplex virus 1 (HSV-1) has been reported.

Extracts of the leaves and roots of Sarracenia plants have been demonstrated to have anti-tumor activity against leukemia cells, epidermoid carcinoma of the nasopharynx, and other types of cancer [7, 8]. Specific compounds that are present in Sarracenia flava extracts, namely, lupeol and betulin, exhibit anti-tumor activity against various types of cancer cells including melanoma [9-11].

Extracts of Sarracenia can contain anthocyanins and their glucosides including pelargonidin, pelargonidin 3-glucoside, cyanidin, cyanidin 3,5-diglucoside, cyanidin 3-glucoside monoglucuronide, peonidin, delphinidin, malvidin, and quercetin. Anthocyanins are the largest group of water-soluble pigments in the plant kingdom and are considered antioxidants, or free radical scavengers. Anthocyanins exhibit several layers of pharmacological activity including induction of programmed cell death in infected or cancerous cells (i.e., apoptosis) while reducing inflammation and inhibiting tumor cell angiogenesis. Sarracenia extracts can also contain 1,4-napthoquinone derivatives: plumbagin, juglone, and menadione, which exhibit strong antioxidant and cytotoxic activities.

The scientific and clinical studies of Sarracenia to date have largely been isolated studies. Despite the research behind the potential for therapeutic uses of Sarracenia extracts, in vitro screening of particular formulations for demonstrating bioactivity in the laboratory is lacking. Pharmaceutical development and validation of specific botanical formulations and their effectiveness for particular indications is therefore important.

### SUMMARY OF THE INVENTION

The invention pertains to compositions and methods of use of Sarracenia-based plant topical therapies for modulating the immune system in cancer, viral infections, and other diseases. Preferred embodiments of the invention pertain to formulations and uses of Sarracenia extracts combined with lidocaine formulated for topical administration for the treatment of skin disorders including wound healing, for the administration of intradermal vaccines, and/or as analgesics.

### BRIEF DESCRIPTION OF THE DRAWINGS

FIG. 1 is a bar graph showing the results of MTT assays testing a Sarracenia formulation for improving cell viability in a model of wound healing in reconstructed human epidermis.

FIG. 2 is a bar graph depicting the results of cytokine production elicited by a Sarracenia formulation in a model of wound healing in reconstructed human epidermis.

FIG. 3A depicts a bar graph showing of the effects of a Sarracenia formulation on surface marker CD40 expression on dendritic cells.

FIG. 3B depicts a bar graph showing of the effects of a Sarracenia formulation on surface marker CD80 expression on dendritic cells.

FIG. 3C depicts a bar graph showing of the effects of a Sarracenia formulation on surface marker CD83 expression on dendritic cells.

FIG. 3D depicts a bar graph showing of the effects of a Sarracenia formulation on surface marker CD86 expression on dendritic cells.

FIG. 3E depicts a bar graph showing of the effects of a Sarracenia formulation on surface marker HLA-DR expression on dendritic cells.

FIG. 3F depicts a bar graph showing of the effects of a Sarracenia formulation on surface marker CCR7 expression on dendritic cells.

FIG. 4 is a bar graph showing IL-8 production by THP-1 cells in the presence of a Sarracenia formulation and TLR agonists.

FIG. 5 is a bar graph showing TNF-alpha production by THP-1 cells in the presence of a Sarracenia formulation and TLR agonists.

FIG. 6A is a bar graph showing the effect of a Sarracenia formulation on adhesion of NCTC2544 (human skin) cells in vitro after 30 minutes.

FIG. 6B is a bar graph showing the effect of a Sarracenia formulation on adhesion of NCTC2544 (human skin) cells in vitro after 60 minutes.

FIGS. 7A-7E are photographs depicting the effect of a Sarracenia formulation in an in vitro wound scratch healing assay. FIG. 7A is representative of the initial scratch (t=0), FIG. 7B shows the carrier control. FIGS. 7C shows the scratch healing of the following concentration of Sarracenia: 0.1 mg/ml. FIG. 7D shows the scratch healing of the following concentration of Sarracenia: 0.5 mg/ml. FIG. 7E shows the scratch healing of the following concentration of Sarracenia: 1.0 mg/ml.

FIG. 8 is a bar graph depicting the effect of a Sarracenia formulation in an in vitro wound scratch healing assay.

FIG. 9 is a bar graph showing the influence of a Sarracenia formulation in a 3D model of wound healing.

### DESCRIPTION OF THE INVENTION

### Definitions

In the context of the present invention, “subject” or “individual” refers to a human (e.g. a subject or an individual with a viral infection, cancer, or skin wound or lesions) and, more broadly speaking, may include any mammal. Accordingly, aspects of the present invention may be practiced in human clinical medicine as well as in veterinary medicine.

In the context of the present invention, the phrase “augmentation of efficacy” or “improved efficacy” of a therapeutic agent includes enhancement of one or more of the therapeutic effect(s) of said therapeutic agent. In the context of the present invention, the therapeutic effect of a drug may refer to inhibition of viral replication, reversal of virus-mediated immune suppression, augmentation of anti-viral or anti-tumor immunity, or promotion of wound healing or skin re-epithelialization, reduction in the appearance and numbers of skin lesions, or reduction of pain.

In the context of the present invention, “augmentation of efficacy” or “improved efficacy” of a therapeutic agent, specifically of an anti-cancer agent, may refer to one or more of the following activities: suppression of tumor growth, induction of tumor regression, suppression of metastasis, inhibition of metastasis, suppression of proliferation, induction of programmed and/or non-programmed cell death, and the like

The term “botanicals” or “botanical ingredients” refers to one or substances derived from a plant. Said substances may be extracts of specific plant components, i.e. components of a specific part of a plant that have been extracted and are dissolved in a carrier.

The term “controlled release” is used herein to refer to pharmaceutical formulations that are combined with ingredients to alter their dissolution profile. A “sustained release” formulation refers to a type of controlled release formulation in which the ingredients are added to a pharmaceutical formulation for which the dissolution profile of the active ingredient is extended over a longer period of time than that of an otherwise comparable immediate-release formulation owing to the presence of suitable excipients configured to control the dissolution profile.

The term “therapeutically effective amount” is used herein in its ordinary sense and thus includes dose units of a specific ingredient or of a formulation of ingredients that, when administered as part of a regimen, provide therapeutic benefit in the treatment of a condition or disorder for which the ingredient or formulation of ingredients is indicated.

In the context of the present invention, “inflammation” refers to the immune system's response to harmful stimuli, such as pathogens, damaged cells, toxic compounds, or irradiation. At the tissue level, inflammation is characterized by redness, swelling, heat, pain, and loss of tissue function, which result from local immune, vascular and inflammatory cell responses to infection or injury. Important microcirculatory events that include vascular permeability changes, leukocyte recruitment and accumulation, and the release of cytokines and other inflammatory mediators.

In the context of the present invention, “Sarracenia extracts” or “formulations of Sarracenia extracts” may refer to one or more of the following unless otherwise indicated: Sarracenia leucophylla, Sarracenia alata, Sarracenia flava, Sarracenia jonesii, Sarracenia minor, Sarracenia oreophila, Sarracenia psittacina, Sarracenia purpurea, Sarracenia rosea, and Sarracenia rubra.

In the context of the present invention, the term “cancer” broadly refers to one or more of the following types of cancer including but not limited to the following: brain, melanoma, bladder, breast, cervix, colon, rectal, head and neck, kidney, lung, non-small cell lung, mesothelioma, ovary, prostate, sarcoma, stomach, uterus, as well as carcinomas, including spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, carcinoma villosum, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypemephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, naspharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, and carcinoma scroti, The term “sarcoma” generally refers to a tumor which is made up of a substance like the embryonic connective tissue and is generally composed of closely packed cells embedded in a fibrillar, heterogeneous, or homogeneous substance. Sarcomas include, chondrosarcoma, fibrosarcoma, lymphosarcoma, melanosarcoma, myxosarcoma, osteosarcoma, endometrial sarcoma, stromal sarcoma, Ewing's sarcoma, fascial sarcoma, fibroblastic sarcoma, giant cell sarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilns' tumor sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, and telangiectaltic sarcoma.

In the context of the present invention, an “immune response modifier” may be any compound that modifies an immune response that is generated in response to a stimulus. One example of an immune response modifier known in the art is Resiquimod (R-848; S-28463; 4-amino-2-ethoxymethyl-α,α-dimethyl-1H-imidazo [4,5-c]quinoline-1-ethanol). Resiquimod is a topically active immune response modifier that can reduces recurrences of herpes after therapy in the guinea pig when administered as infrequently as once weekly. Resiquimod has no direct antiviral activity but induces endogenous production of interferon (IFN)-α and interleukin-12 directly, IFN-γ indirectly, and enhances dendritic cell (DC) antigen presentation. Broadly speaking, many imidazoquinoline amine, imidazopyridine amine, 6,7-fused cycloalkylimidazopyridine amine, and 1,2-bridged imidazoquinoline amine compounds have been classified as immune response modifiers.

Another example of an immune response modifier is imiquimod, a synthetic imizoquinoline compound which can act on Toll-like receptor (TLR)-7 and transduce signals involved in cell activation. Imiquimod is applied topically as a 5% cream for treating superficial basal cell carcinoma, actinic keratosis, genital warts, and other skin conditions. When applied, imiquimod leads to an inflammatory reaction that induces regression of skin neoplasms and virus-induced papillomas. However, adverse events may be associated with this topical agent commonly include application site irritation, primarily erythema, oozing, and erosions, as well as systemic effects including headache, sinusitis, myalgia, and fatigue. In treatment of basal cell carcinoma, meta-analyses have reported that more patients who are treated with imiquimod have adverse events as compared to other treatments for this condition [12].

In terms of their activities, immune response modifiers are compounds that have demonstrated potent immunostimulating, antiviral, antitumor activities. Immune response modifiers may also be useful as vaccine adjuvants to enhance protective immune system response to vaccines; for example, as adjuvants for cancer vaccines. Many such compounds are disclosed in, for example, U.S. Pat. Nos. 4,689,338, 5,389,640, 5,268,376, 4,929,624, 5,266,575, 5,352,784, 5,494,916, 5,482,936, 5,346,905, 5,395,937, 5,238,944, and 5,525,612, WO 93/20847. However, in the context of this invention, it is also understood that immune response modifiers also have the capacity for changing both pro-inflammatory classical T helper-1 (Th1) type responses that are associated with anti-viral and anti-tumor immunity, but also for modulating the T-helper 2 responses that might counteract the former responses.

### Embodiments of the Invention

This invention discloses formulations comprising extracts of pitcher plants for one or more of the following uses: a) anti-viral agent; b) anti-tumor agent; c) wound healing; d) analgesic; e) adjuvant for therapeutic vaccines. In preferred embodiments, said pitcher plants are Sarracenia.

The present invention discloses formulations comprising Sarracenia extract. Embodiments of the invention include extracts preferably from one or more of the following: Sarracenia leucophylla, Sarracenia alata, Sarracenia flava, Sarracenia jonesii, Sarrcenia minor, Sarracenia oreophila, Sarracenia psittacina, Sarracenia purpurea, Sarracenia rosea, and Sarracenia rubra. Preferred embodiments of the invention are formulations comprising Sarracenia leucophylla.

In a preferred embodiment, formulations comprised of extracts of Sarracenia are derived from one or more of the following plant components: a) flower; b) leaf; c) seed; d) stalk. Specific embodiments comprise a composition that contains Sarracenia extracts derived from the flower, leaf, seed, and stalk that are combined together in said formulation. The extracts may be obtained by methods known to one of ordinary skill in the art.

Additional compounds can be added to the Sarracenia extracts during the formulation process. For example, extracts of other botanicals that have effects on one or more of the following: a) anti-viral agent; b) anti-tumor agent; c) wound healing; d) pain; and/or, e) adjuvant for therapeutic vaccines.

Topical analgesics may be added to the Sarracenia extracts during the formulation process including but not limited to compositions of capsaicin, diclofenac sodium, lidocaine, methyl salicylate and menthol, trolamine (Aspercreme).

Other topical analgesics known in the art may be combined with the formulations disclosed herein to achieve enhanced pain relief and tissue healing including but not limited to: pramoxine, benzocaine, dibucaine, prilocaine, and tetracaine.

The present compositions may also include or bases, polymers, coplymers, emulsifiers, binders, and tissue-soothers known in the art (for example, Vitamin A, Vitamin E, cocoa butter, oils/creams, glucononolactone, DMSO, sodium hyaluronate, hyaluronic acid, lecithin, glycerin, water, ammonium acryloyldimethyltaurate/VP copolymer, aloe vera, edetate disodium, allantoin and methylchloroisothiazolinone/methylisothiazolinone, pelargonidin, pelargonidin 3-glucoside, cyanidin, cyanidin 3,5-diglucoside, cyanidine 3-glucoside monoglucuronide, peonidin, delphinidin, malvidin, quercetin, related anthocyanins and their glucosides); and preservatives known to those skilled in the art.

In some embodiments, said formulations comprising extracts of Sarracenia can be formulated for topical application to the skin. In other embodiments, suppositories can be formulated for use in vaginal applications. The suppositories may contain between 0.1-5% of Sarracenia extract.

The formulations comprising Sarracenia extracts disclosed herein can be formulated as a liquid extract (also called a tincture). For example, the present composition is formulated with a fatty acid base that holds the tincture of Sarracenia extracts. One example of such a base is lecithin, which can facilitate emulsification of the water-extracted constituents of Sarracenia derived using methods well-known to one of ordinary skill in the art of preparing said extracts. The pH of the Sarracenia extract will be maintained using methods known in the art to ensure that plant constituents can remain active when applied topically.

In a preferred embodiment of the invention, the composition of Sarracenia extracts is formulated into a gel for topical application to human tissues. One embodiment may involve the composition of Sarracenia extracts in a versabase gel. In other embodiments, said composition is formulated as a cream or a lip balm.

In another embodiment, the composition of Sarracenia extracts is formulated into a transdermal patch or any other adhesive that is placed on the skin to deliver a specific dose of medication through the intradermal route.

In another embodiment, the formulations disclosed herein comprising Sarracenia extracts are formulated for local injections into a subject in need thereof.

In another embodiment, the formulations disclosed herein comprising Sarracenia extracts are formulated for delivery as a medicated spray.

Preferred embodiments of the formulation comprising Sarracenia extracts also comprise the following ingredients and compounds: Aloe barbadensis leaf juice, glycerin, alcohol, carbomer, phenoxyethanol, allantoin, lidocaine HCl, capryly glycol, sodium hydroxide, Lavandula angustifolia flower extract, ethylhexylglycerin, hexylene glycol, dipotassium glycyrrhizate, Melissa officinalis extract, Melissa officinalis leaf extract, Glycyrrhiza glabra root extract, Hypericum perforatrum flower/left extract, Camellia sinensis leaf extract, Acanthopanax senticosus root extract, sodium lauroyl lactylate, ceramide NP, ceramide AP, phytosphingosine, cholesterol, xanthan gum, and ceramide EOP.

In one embodiment of the invention, the formulation comprises Sarracenia leucophylla flower, leaf, seed, and stalk extracts at approximately 0.165% wt/wt and also Aloe barbadensis leaf juice at approximately 10.0% wt/wt, Lavandula angustifolia extract at approximately 0.156% wt/wt, Melissa officinalis extract at approximately 0.100% wt/wt, Melissa officinalis leaf extract at approximately 0.0565% wt/wt, Glycyrrhiza glabra root extract at approximately 0.0320% wt/wt, Hypericum perforatum flower/leaf extract at approximately 0.0250% wt/wt, Camellia sinensis leaf extract at approximately 0.0215% wt/wt, and Acanthopanax senticosus root extract at approximately 0.0180% wt/wt.

Certain embodiments of the invention comprise Sarracenia leucophylla flower, leaf, seed, and stalk extracts at approximately 0.1-0.6% wt/wt, Aloe barbadensis leaf juice at approximately 5-15% wt/wt, Lavandula angustifolia extract at approximately 0.1-0.6% wt/wt, Melissa officinalis extract at approximately 0.05-0.50% wt/wt, Melissa officinalis leaf extract at approximately 0.01-0.1% wt/wt, Glycyrrhiza glabra root extract at approximately 0.01-0.06% wt/wt, Hypericum perforatum flower/leaf extract at approximately 0.01-0.06% wt/wt, Camellia sinensis leaf extract at approximately 0.01-0.06% wt/wt, and Acanthopanax senticosus root extract at approximately 0.01-0.06% wt/wt.

In another embodiment, the formulation comprises botanicals, allantoin, and lidocaine.

In one embodiment, said botanicals include Sarracenia stalk, flower and seed extract, Sarracenia Purpurea leaf, stalk, flower, and seed extract, Aloe Barbadensis leaf juice, Lavandula Angustifolia (lavender) flower extract and Melissa Officinalis extract.

In one embodiment, the composition contains allantoin 0.50% (W/W). In other embodiments, said composition may contain allantoin between 0.25-2% (W/W). In other embodiments, the composition includes lidocaine 0.50% (W/W). In other embodiments, said composition may contain allantoin between 0.25-2% (W/W)

In a preferred embodiment, said formulation containing Sarracenia extract is comprised of a gel for topical administration directly to a skin lesion, wound, tumor, or site of irritation to a subject in need thereof.

Preferred embodiments of the invention comprise the following: a) Sarracenia leucophylla flower, leaf, seed, and stalk extracts; b) Lidocaine HCl; and, c) Other botanical ingredients. Said other botanical ingredients may comprise extracts derived from one or more of the following plants: a) Aloe barbadensis; b) Lavandula angustifolia; c) Melissa officinalis extracts; d) Glycyrrhiza glabra; e) Hypericum perforatum; f) Camellia sinensis; and, g) Acanthopanax senticosus. Said combination of ingredients administered in therapeutically effective amounts may be used to provide synergistic effects in managing pain and inflammation when applied topically to the skin or via other routes of administration or to other tissues. Said combination of ingredients may also be administered in therapeutically effective amounts to provide immune balancing or immune modulating effects. The immune system must effectively regulate the balance between beneficial and detrimental inflammation. While an inflammatory response is necessary, for example, to eradicate pathogens, it must also be regulated in terms of its intensity and duration in order to prevent tissue damage. To this end, lidocaine may provide analgesic effects while Sarracenia extracts disclosed herein may stimulate inflammation-resolving mediators, which may be produced by immune cells of the DC, myeloid cell, T cell or B cell lineages, and the like. Therefore, in some embodiments, a formulation comprising Sarracenia extract lidocaine HCl, and also comprising additional botanicals, may be used for indications where symptoms of inflammation characterized by tissue redness, pain are present. Such symptoms typically arise in response to localized protective reactions of tissue against irritation, infection, injury, allergy, and tumors.

In one specific embodiment of the invention, the formulation comprises Sarracenia leucophylla flower, leaf, seed, and stalk extracts at approximately 0.165% wt/wt, lidocaine HCL at 0.5% wt/wt, Aloe barbadensis leaf juice at approximately 10.0% wt/wt, Lavandula angustifolia extract at approximately 0.156% wt/wt, Melissa officinalis extract at approximately 0.100% wt/wt, Melissa officinalis leaf extract at approximately 0.0565% wt/wt, Glycyrrhiza glabra root extract at approximately 0.0320% wt/wt, Hypericum perforatum flower/leaf extract at approximately 0.0250% wt/wt, Camellia sinensis leaf extract at approximately 0.0215% wt/wt, and Acanthopanax senticosus root extract at approximately 0.0180% wt/wt.

The formulations disclosed herein are suitable for topical administration, that be formulated as an ointment, cream, suspension, lotion, powder, solution, paste, gel, spray, aerosol or oil. Alternatively, said formulations can comprise a patch or a dressing such as a bandage or adhesive plaster impregnated with active ingredients, and optionally one or more excipients or diluents. Topical formulations preferably comprise compounds that facilitate absorption of the active ingredients through the skin and into the bloodstream. Topical formulations may also include a suitable excipient configured to control the dissolution profile of the active ingredients for the purpose of controlled-release or sustained-release of said active ingredients comprising the formulation.

Compositions of the present invention may be liquids or lyophilized or otherwise dried formulations and include diluents of various buffer content (e.g., Tris-HCl, acetate, phosphate), pH and ionic strength, additives such as albumin or gelatin to prevent absorption to surfaces, detergents (e.g., Tween®, Pluronic®, bile acid salts), solubilizing agents (e.g., glycerol, polyethylene glycerol), anti-oxidants (e.g., ascorbic acid, sodium metabisulfite), preservatives (e.g., Thimerosal™, benzyl alcohol, parabens), bulking substances or tonicity modifiers (e.g., lactose, mannitol), covalent attachment of polymers such as polyethylene glycol to the protein, complexation with metal ions, or incorporation of the material into or onto particulate preparations of polymeric compounds such as polylactic acid, polyglycolic acid, hydrogels, etc., or onto liposomes, microemulsions, micelles, unilamellar or multilamellar vesicles, erythrocyte ghosts, or spheroplasts. Such compositions will influence the physical state, solubility, stability, rate of in vivo release, and rate of in vivo clearance. Controlled or sustained release compositions include formulations in lipophilic depots (e.g., fatty acids, waxes, oils).

The formulations described herein can be utilized in conjunction with previously known herpes medications or other wound healing compounds and treatments. Examples of known herpes medications include Allantoin and Lidocaine, these can be used in conjunction with or in substitution for acyclovir, famciclovir, valacyclovir, docosanol, or lysine, for example.

This invention teaches methods for prevention and treatment of skin disorders using the formulations disclosed herein comprising Sarracenia extracts. This invention also teaches methods and compositions for preventing, improving or alleviating skin disorders caused by viral infections, bacteria or fungal infections, neoplasms, wounds, acne, rosacea, autoimmune disorders, and/or allergies in a subject in need thereof.

This invention teaches methods and compositions for treatment of viral infections using a formulation containing Sarracenia extracts. In a preferred embodiment, the invention teaches the treatment of herpes simplex virus (HSV) using a topically applied formulation of Sarracenia extract.

In another embodiment, compositions of Sarracenia extracts are disclosed for the treatment of orofacial manifestations of HSV-1 (i.e. cold sores; herpes labialis). In one embodiment, formulations comprising Sarracenia extracts disclosed herein are used as an anti-viral treatment for the prevention of replication of HSV-1 by administration of said formulations topically to the affected area of skin. Specifically, formulations comprising Sarracenia are used for accelerating skin healing, reducing pain and/or for reducing the appearance of lesions resulting from HSV-1 infection. Said formulations may be used to reduce the sensation of pain, appearance or severity of blisters and sores, and to mitigate symptoms such as fever, sore throat and enlarged lymph nodes. Said formulation may also be used prophylactically for prevention of the symptoms of herpes labialis or to reduce their severity.

Recurrence of orofacial HSV-1 infection due to reactivation of the virus is a complication that is associated with chemical peeling procedures of skin. In another embodiment, compositions of Sarracenia extracts in therapeutically effective amounts are used prophylactically by topically administering said formulations prior to a peeling procedure and/or after completion of peeling procedure.

In one embodiment, formulations comprising Sarracenia extracts are used for the prevention or treatment of HSV-2 infections. Said infections may be manifested as sores or lesions that are sexually transmitted.

The formulations disclosed herein may be used for the treatment of manifestations of infection with Varicella zoster virus, the causative agent of chickenpox.

The formulations disclosed herein may also be applied to skin sites following the performance of a skin biopsy.

Basal cell carcinomas (BCCs) and squamous cell carcinomas (SCCs) of the skin are the most common malignancies in fair-skinned humans, and their incidence is increasing. The prevalence of actinic keratoses (i.e., premalignant intra-epidermal keratinocyte neoplasias), which can develop into invasive SCC, is also high. In one embodiment of the invention, formulations of Sarracenia extract disclosed herein are used for the treatment of BCCs or SCCs. In preferred embodiments, a topical formulation of said Sarracenia extract is administered directly to the surface of the tumor. In other cases, said formulations may be administered to the site prior to or following surgical excision of the tumor. Alternatively, said formulations may be administered in a therapeutic regiment with 5% imiquimod cream and/or with photodynamic therapy. Said formulations may be administered directly following a tissue biopsy of skin to identify a suspected malignant lesion. The surgical procedure to obtain the biopsy may induce local bleeding and bruising, pain, infection, allergic reaction to the numbing medicine used in the procedure, or damage to the structures beneath the skin site, which may be mitigated or reduced by formulations described herein.

In one embodiment of the invention, formulations comprising Sarracenia extracts disclosed herein are used to modify the activity of DC; specifically, to induce characteristics in DC that are conducive to the maturation and/or activation of these cells. When DC are stimulated in vivo by an activating stimulus, they undergo maturation and migrate to lymphoid organs where they activate several effector cells of the immune system, primarily T-cells and B-cells. These immunostimulatory activities of DC can lead to anti-tumor or anti-viral immunity, for example. During maturation, DC also upregulate their expression of specific molecules including CD83, enhanced HLA class I and II molecules, increased expression of costimulatory molecules such as CD40, CD80, and CD86), as well as gaining the capability to secrete high levels of biologically active cytokines such as IL-12 p70.

In embodiments of the present invention, the formulations comprising Sarracenia extracts described herein are used for generating immunostimulatory DC and/or to enhance the maturation of DC. It is known in the art that DC are frequently generated in vitro for research and clinical laboratories using starting populations of monocytes, peripheral blood mononuclear cells, bone marrow cells, or the like. In other cases, DC are generated from cell lines; for example, using a starting population of THP-1 cells (human monocytic cell line derived from an acute monocytic leukemia patient). The starting cell populations are typically cultured in recombinant cytokines such as granulocyte-macrophage colony stimulating factor (GM-CSF), interleukin 4 (IL-4) to induce the generation of immature DC, and subsequently, also to DC maturation agents such as tumor necrosis factor alpha (TNF-alpha), ionophores (i.e. ionomycin) and/or TLR agonists. It is known to one skilled in the art that there are many protocols for generating DC in vitro with variations in the specific reagents used, their concentrations added to the cultures, and durations of culture for DC generation from myeloid-lineage cells and their subsequent maturation. Many reviews have been published on the generation of human DC and DC from other mammals, particularly murine (a non-limiting example describing basic protocols for DC generation is provided in [13]). The phenotypes and immunostimulatory capabilities of DC generated via different protocols may also exhibit variability, the impact of which is determinable based on the intended use for the DC. In the context of the present invention, said cultured cells grown using one or more of these protocols may be exposed to a formulation comprising Sarracenia extracts disclosed herein during the process of generating DC in vitro or after completion of the culture protocol being implemented for generating mature DC. The formulations disclosed herein may be added directly to the culture medium in which the cells are being generated.

In preferred embodiments, the formulations comprising Sarracenia extracts are administered to cultures with maturing DC at concentrations of 500-2,500 μg/mL or, more preferably, 1,000 μg/mL.

Subsequently, in order to evaluate DC that were exposed to said formulation comprising Sarracenia extract, flow cytometry may be performed to evaluate expression levels of HLA molecules, and costimulatory molecules (CD80, CD86, CD40, and others). Production of specific cytokines by DC may also be evaluated using techniques such as ELISA, ELISpot, and other analytical methods for cytokines that are known in the art. Additionally, the DC that have been exposed to the formulations disclosed herein may be co-cultured with T cells and evaluated for their capabilities to elicit typical molecules known to be associated with T cell activation. In all of the aforementioned experimental scenarios, DC that have been exposed to one of the formulations of Sarracenia disclosed herein may be compared to DC exposed to a vehicle control of said formulation that does not contain the active Sarracenia ingredient.

In one specific embodiment, the compositions of Sarracenia extracts disclosed herein are applied for the purpose of modifying expression at the protein level of human leukocyte antigen (HLA) molecules on one or more cell types. HLA molecules that are affected by the formulations disclosed herein may include classical and non-classical HLA molecules known in the art including class Ia (HLA-A, -B, -C), class lb (HLA-E, -F, -G, -H), and class II (HLA-DR, -DQ, -DM, and -DP).

In one specific embodiment, the compositions of Sarracenia extracts disclosed herein are applied for the purpose of modifying expression of killer cell immunoglobulin-like receptors (KIRs) on natural killer (NK) cells; for example, KIR, NKG2A/CD94, ILT2, and LIR1.

DC are cells that are uniquely potent in their ability to present antigens to T cells, and this property has prompted their recent application to therapeutic cancer vaccines. Isolated DCs loaded with tumor antigen ex vivo and administered as a cellular vaccine have been found to induce protective and therapeutic anti-tumor immunity. Sipuleucel-T (Provenge), which is approved for the treatment of advanced prostate cancer, is an example of a DC vaccine.

The formulations described herein may provide an improved cell manufacturing method for generating DC in clinical labs or for research purposes. Many of the culture protocols for generating DC inadvertently generate inhibitory signals instead of the favorable immunostimulatory signals. If DC are highly stimulated ex vivo, they can undergo “exhaustion” or “paralysis” and will not function for activating immunity [14]. Ex vivo-generated DC that are too mature or too highly stimulated may also fail to migrate to the lymph nodes upon injection. On the other hand, DC that are too immature will not have the capacity to activate T cell immune responses upon injection.

In one embodiment, the formulations comprising Sarracenia extracts disclosed herein are used for generating autologous DC ex vivo, where said DC are then used for immunizing individuals with cancer as a form of immunotherapy. The DC may be washed to remove the formulations comprising Sarracenia extracts prior to administering said DC as immunotherapy. Alternatively, the formulations comprising Sarracenia extracts may be co-administered with DC to an individual in need thereof.

The formulations described herein may be used to modify the responses of cells to TLR agonists or antagonists in cell culture. Examples of cells that may be modulated by the formulations comprising Sarracenia extracts include DC, macrophages, other cells of the immune system, peripheral blood leukocytes, and epithelial cells. For example, the formulations disclosed herein may be used to modulate cytokine production by cells responding to TLR agonists or antagonists. In this context, the invention may be used as a tool in a research laboratory or for generating immunotherapeutic cells for clinical workflows.

The formulations described herein may be used to modify the responses of cells to TLR agonists or antagonists in vivo. In one embodiment, the formulations comprising Sarracenia extracts disclosed herein may be used as immune response modifiers. One manner said modulation of immune responses can be gauged is by measurement of cytokines using methods known in the art. The cytokines capable of modulation by the formulations described herein include IL-2, IL-4, IL-6, IL-8, IL-10, IL-12, IL-17, IL-18, IL-27, IL-33, IL-37, TNF-alpha, IFN-gamma, other interferons, G-CSF, M-CSF, and GM-CSF.

Alternatively, said formulations may be used as adjuncts to immune response modifiers that are known in the art, examples being Resiquimod and Imiquimod.

In one embodiment, formulations comprising Sarracenia extracts disclosed herein are co-administered in therapeutically effective amounts in combination with Aldara (imiquimod). These agents are preferably both administered topically. In one example, for the treatment of warts, a thin layer of Aldara combined with a topical formulation comprising Sarracenia extracts described herein is applied to the affected area of skin once daily 3 times per week on nonconsecutive nights just prior to sleep and is washed off in the morning. This therapeutic regimen is continued until warts are cleared. In a related example, Aldara may be administered as described and topical formulations comprising Sarracenia extracts are applied to an individual in need thereof once daily 4 times per week and on the nights that Aldara is not given. The co-application of formulations comprising Sarracenia extracts may be used to improve the healing time for warts or to improve the response rates to Aldara (for example, in immunosuppressed individuals). The formulations disclosed herein may also be used in combination with Aldara for the treatment of actinic keratosis, basal cell carcinoma, and/or external genital and perianal warts resulting from human papilloma virus (HPV).

This invention also discloses methods for improving the immunogenicity of cancer vaccines. In one embodiment, the formulations comprising Sarracenia extracts disclosed herein are used as adjuvants for immunotherapy. In preferred embodiments, said formulations are administered topically to the site where an injection comprising a vaccine is to be administered intradermally just prior to the administration of said vaccine. Said formulations comprising Sarracenia extracts may be administered alone or in combination with another topical immune response modifier (e.g. imiquimod).

In one example, formulations comprising Sarracenia extracts in a gel or cream form are self-applied in therapeutically effective amounts by an individual in need thereof to a 45-cm outlined area of healthy skin overnight on days 1-5 of a treatment cycle for cancer vaccination. Application and removal times are recorded in treatment diaries, Subsequently, a cancer vaccine is injected intradermally into the same site on day 3. These cycles are repeated, for example, every 3 weeks. Said formulations comprising Sarracenia extracts may be administered alone or in combination with another topical immune response modifier (e.g. imiquimod). Subsequently, systemic immunity of both humoral and cellular types can be measured to measure the induction of vaccine-specific immunity and/or T helper-1 immunity that has been induced by using formulations comprising Sarracenia extracts as adjuvants.

In another embodiment, compositions of Sarracenia extract are used as pain relievers or analgesics. Formulations described herein for use as analgesics may comprise at least two of the following: a) Lidocaine; b) Sarracenia extracts; c) Botanicals ingredients consisting of extracts of at least one of the following: Aloe barbadensis, Lavandula angustifolia, Melissa officinalis extracts, Glycyrrhiza glabra, Hypericum perforatum, Camellia sinensis; and, Acanthopanax senticosus. Use of lidocaine in combination with either the formulations of Sarracenia extracts and/or combinations of the botanicals disclosed herein may be applied topically for pain relief to provide synergistic improvements in pain relief over lidocaine alone and also to provide simultaneous healing of wounds, lesions or tissues.

In one embodiment, compositions of Sarracenia extract are used for inhibiting neuromuscular or neuralgic pain.

In another embodiment, compositions of Sarracenia extract are used to provide symptomatic relief and/or to slow progression of diabetic neuropathy.

In another embodiment, compositions of Sarracenia extract are used for treatment of atopic dermatitis, specifically, for the prevention, reduction or treatment of lesions, rashes itching, inflammation, eczema, and secondary infections of the skin.

Said compositions of Sarracenia extract may also be used for the treatment of contact dermatitis for the alleviation of symptoms of redness, itching, swelling, blistering, and eczema of the skin. Use of the formulations disclosed herein may include irritant dermatitis, phototoxic dermatitis, allergic dermatitis, photoallergic dermatitis, contact urticaria, systemic contact-type dermatitis and the like.

This invention teaches formulations and methods for preventing, reducing or ameliorating the symptoms caused by skin irritants when administered in therapeutically effective amounts to an individual in need thereof. Skin irritants may include common household items that certain individuals develop reactions to including but not limited to soap, household cleaners, fabric dryer sheets, latex, cosmetic products, pollen, plastics, textiles, and metals (e.g. nickel, cobalt, chromium).

This invention also teaches formulations and methods for prevention and treatment of autoimmune disorders; specifically, those conditions affecting the skin including epidermolysis bullosa acquista, IgA-mediated bullous dermatoses, pemphigoid, pemphigus and/or Lichen planus.

In another embodiment, compositions of Sarracenia extract are used for the treatment of ichthyosis or xeroderma. Said compositions may be administered for reducing itching and irritation.

In another embodiment, compositions of Sarracenia extract are used for the treatment of seborrheic dermatitis. Said compositions may be administered for reducing or preventing inflammatory scaling present on the skin.

In other embodiments, compositions of Sarracenia extract are used for the treatment of seborrheic dermatitis, contact hypersensitivity, psoriasis, cutaneous eosinophilias, angiodemas, vasculitides, erythemas, uticaria, dermatitis herpetiformis, psoriasis, candidiasis, and/or ichthyosis vulgaris.

The formulations described herein may also be used to treat other skin disorders or pathologies. Embodiments of the invention include use of the formulations described herein for treating and/or alleviating symptoms of acne, rosacea, diaper rash, eczema and skin damage from a variety of causes including wounds, burns, fistulas, and fecal and urinary incontinence.

The cellular regeneration of the skin is maintained by different adult stem/progenitor cell subpopulations localized within the specialized microenvironments, niches in interfollicular epidermis, sebaceous gland and hair follicle bulge region. The study of artificial skin reparation involves the application of modifying materials which cover the wound for temporary protection of the wound surface, and now, has reached to induction of more aggressive treatments by using agents such as cytokines and growth factors that are related to wound healing. There remains a need for novel agents to induce epithelialization that can be useful for regeneration and repair of various degrees of skin wounds, from simple to refractory, including regeneration of the hypodermis.

This invention teaches formulations and methods for skin regeneration and re-epithelization. In vivo, the formulations of Sarracenia extracts disclosed herein may be applied for modulating inflammation, reducing fibrosis, and mobilizing stem cells to sites of skin damage. The formulations disclosed herein may be applied for is therefore applicable to skin grafting, anti-aging therapies, and for regeneration of the skin using stem cell administration. In one embodiment, the formulations of Sarracenia extracts disclosed herein may be incorporated into the matrix of skin substitutes or in wound dressings that are known in the art for the purpose of treating burns or chronic, slow healing wounds.

The formulations described herein may also be used as a component of treatment for Staphylococcal skin infections. These infections can range from minor skin infections to life-threatening infections that may cause boils, impetigo, cellulitis, and staphylococcal scalded skin syndrome. In one embodiment, Sarracenia formulations described herein are used to reduce pain and inflammation, and/or to improve healing of lesions. The formulations described herein may also be used as a component of treatment for Staphylococcal skin infections as an adjunct to therapies (i.e., antibiotics).

It is known in the art that there is a continuing need for development of new and improved, nontoxic antipruritic and pH-adjusting agents that are effective in treating and alleviating skin disorders, pathologies and pruritus resulting from a wide variety of causes or causes different than those that can be treated by currently available agents. In one embodiment, the compositions of Sarracenia extract described herein may be used for the treatment of pruritis. In other embodiments, said compositions of Sarracenia extract may be co-administered with other treatments for pruritis, e.g., hydrocortisone, fluocinide, betamethasone valerate, fluocinolene acetonide, triamcinolone acetonide. Remission of pruritis is often slow and incomplete. Therefore, formulations comprising Sarracenia extract disclosed herein may be applied for enhancing the effectiveness of one or more therapeutic agents used for addressing pruritis.

### EXAMPLES

### Example 1

### Evaluation of the Capacity of the Formulations Comprising Sarracenia Extracts to Repair Skin Irritant-Induced Skin Damage

### Objectives:

Despite the uses of botanical formulations, including Sarracenia extracts, as components of anti-viral compounds for topical applications, there are no data on their capabilities for repairing the damage caused to skin by irritants.

A formulation of Sarracenia comprising the following ingredients shown in Table 1 was evaluated in reconstructed human epidermis (this formulation was used for all examples provided herein):

TABLE 1

| INGREDIENT | % WT/WT |
|---|---|
| Water (Aqua) | 77.7460549% |
| Aloe Barbadensis Leaf Juice | 10.0000000% |
| Alcohol Denat. | 1.7020000% |
| Allantoin | 0.5000000% |
| Lidocaine HCl | 0.5000000% |
| Sarracenia Leucophylla Flower/Leaf/Seed/Stalk Extract | 0.1650000% |
| Lavandula Angustifolia (Lavender) Flower Extract | 0.1565000% |
| Melissa Officinalis (Balm Mint) Extract | 0.1000000% |
| Melissa Officinalis Leaf Extract | 0.0565000% |
| Glycyrrhiza Glabra (Licorice) Root Extract | 0.0320000% |
| Hypericum Perforatum Flower/Leaf Extract | 0.0250000% |
| Camellia Sinensis Leaf Extract | 0.0215000% |
| Acanthopanax Senticosus (Eleuthero) Root Extract | 0.0180000% |

### Methods:

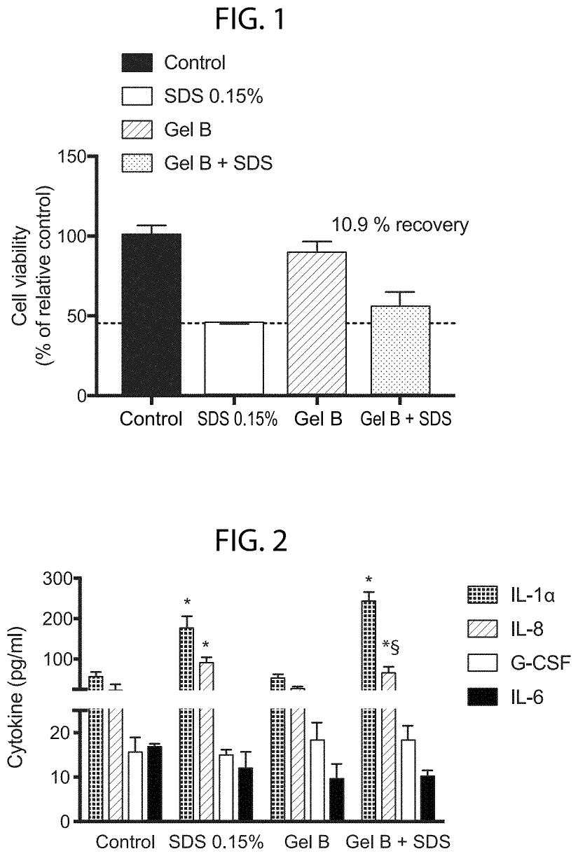

Reconstructed human epithelium (RhE) was damaged by exposure to SDS 0.15% for 1 h, then 25 μL of the final Sarracenia formulation (Gel B) was applied. Cell viability by the MTT assay (FIG. 1), and cytokine release, specifically, IL-1α, IL-6, IL-8 and G-CSF release measured using ELISA (FIG. 2), were evaluated 24 h later. Each treatment was run in triplicate. A statistically significant (Tukey's multiple comparison test) recovery in cell viability and cytokine release compared to SDS treated inserts was considered as indicator of repair capacity.

Each value represents the mean±SD, n=3. **p<0.01 vs control. The dotted line is set at the level of cytotoxicity obtained following SDS damage.

The results of the MTT assay depicted in FIG. 1 show that, as expected, there was significant damage to the RhE induced by SDS as demonstrated by a reduction in cell viability below 50%. Gel B, the complete Sarracenia formulation, did not significantly alter cell viability as compared to the control. Gel B induced a modest recovery of RhE that was exposed to SDS (10.9% recovery of cell viability), a result that was not statistically significant over RhE with SDS alone.

FIG. 1 (legend): Sarracenia extract formulation improve cellular viability in a skin irritant model. The results obtained on cell viability (MTT assay) are reported. Each value represents the mean±SD, n=3. Statistical analysis was performed with Tukey's multiple comparison test with **p<0.01 vs Control. The dotted line is set at the level of cytotoxicity obtained following SDS damage.

In the studies evaluating cytokine production, the results showed that treatment with SDS caused the expected increase in IL-1α and IL-8 release compared to the control group that was untreated. A modest and statistically significant reduction in SDS-induced IL-8 release was observed in the group exposed to SDS+Gel B, suggesting a slight protection by Gel B on SDS-induced damage. No change in IL-6 or G-CSF release was observed among the treatment groups.

FIG. 2 (legend): Evaluation of cytokine production. Each value represents the mean±SD, n=3. Statistical analysis was performed with Tukey's multiple comparison test with *p<0.05 vs Control and § vs SDS treated group.

### Conclusions:

The Sarracenia formulation (Gel B) induced a recovery in SDS-induced skin damage as demonstrated by an increase in cell viability and reduction in IL-8 release, which is anticipated to have a beneficial effect in mitigating injury.

### Example 2

### Assessment of the Effect of Formulations Comprising Sarracenia Extracts on Dendritic Cells

### Objective:

These experiments evaluated the impact of Sarracenia extract on DC. The human promyelocytic cell line, THP-1, was used as experimental model. THP-1 is a widely used model for primary human monocytes/macrophages. THP-1 cells are suitable for the differentiation of immature DCs (iDCs) and mature DCs (mDCs) in terms of phenotypic, morphologic and functional properties of DCs [15].

### Methods:

For DC differentiation, THP-1 cells were treated for 5 days with rhIL-4 and recombinant human (rh) GM-CSF to acquire the properties of immature DC. Mature DCs were then generated from immature DCs by addition of rhTNF-α and ionomycin into the medium for 2 days. Immature DC were also exposed to Sarracenia extract (1000 μg/ml) or the vehicle base to evaluate its effect on DC maturation. As markers of DC maturation CD40, CD80, CD83, CD86, HLA-DR, CCR7 and IL-10, IL-12p40, TGFβ1 production were assessed. Surface markers were evaluated by FACS analysis, while specific ELISA kits were used to measure cytokine and MMP production.

### Results:

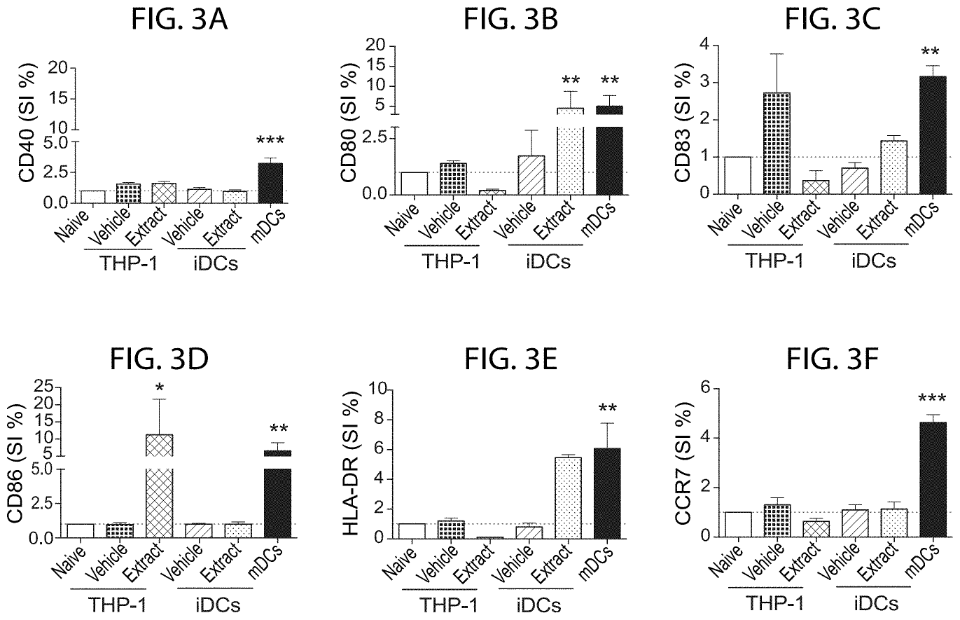

The impact of the Sarracenia formulation on DC maturation was evaluated. Control THP-1 cells were treated with stimuli that are known to induce DC maturation. The results showed that DC maturation occurred as shown by the increases in surface protein expression of typical DC molecules. The exposure of iDCs to the extract was able to induce an increase of CD80 (FIG. 3B) and HLA-DR (FIG. 3E) markers expression, in both cases comparable with mDCs levels. For all the others cell surface markers tested, the extract was not able to induce a statistically significant increase of DCs maturation markers, indicating a partial effect compared to the cocktail used to induce DC maturation. Results are shown in FIGS. 3A-3F.

FIGS. 3A-3F: The Sarracenia formulations induce upregulation of CD80 and HLA-DR by dendritic cells. THP-1 cells were treated for 5 days with rhIL-4 and rhGM-CSF to acquire the properties of immature DCs (iDCs). Mature DCs was then generated from iDCs by addition of rhIL-4, rhGM-CSF, rh-TNF alpha and ionomycin, or treated with Sarracenia extract (1000 μg/ml) or its vehicle for 24 h to evaluate its ability to drive DC maturation. The results show expression levels of surface molecules that are relevant to DC maturation, as analysed by flow cytometry, on THP-1 cells and iDCs under the various treatment conditions.

### Conclusions:

The results indicate that the Sarracenia extract alone was able to induce a significant increase in specific DCs maturation markers (i.e. CD86, and HLA-DR), indicating a partial effect compared to the cocktail used to induce DC maturation. This phenotype is consistent with DC that have properties that are conducive with generating an immune response

### Example 3

### Assessment of the Effect of Formulations Comprising Sarracenia Extracts on Immune Responses Generated by Toll-Like Receptor (TLR) Agonists

The immune response to invading microorganisms such as bacteria and viruses begins with the ligation of Toll Like Receptors (TLRs) on monocytes by TLR agonists that are expressed by the pathogen, which leads to activation of the immune system. One embodiment of the present invention is the use of the Sarracenia extract as an anti-viral agent; therefore, the ability of the formulation to modulate TLR agonist-induced immune responses was evaluated in vitro. The effects of the formulation on immune responses driven by TLR 3, 9, 7/8 agonists were assessed. LPS, a TLR 4 agonist, that typifies bacterial infections was used as a positive control immune stimulant.

### Methods:

Naïve THP-1 cells were exposed for 24 to the TLR3 agonist poly(I:C) (100 μg/ml), to the TLR7/8 agonist imidazoquinoline (1 μg/m1), to the TLR9 agonist Class A CpG oligonucleotide 5 μM, and to LPS from Escherichia coli serotype 0127:B8 10 ng/ml in the presence or absence of three not cytotoxic concentrations of the extract. IL-8 and TNF-α were measured by ELISA as reference cytokines. Three independent experiments will be conducted. A statistically significant difference versus control cells was considered relevant.

### Results:

In the control wells containing TLR-stimulated THP-1 cells, the results showed that Poly(I:C), imidazoquinoline (Imid) and LPS induced IL-8 release. TNF-α release from THP-1 cells was induced only by LPS, while CpG failed to induce IL-8 or TNF-α release (data not shown).

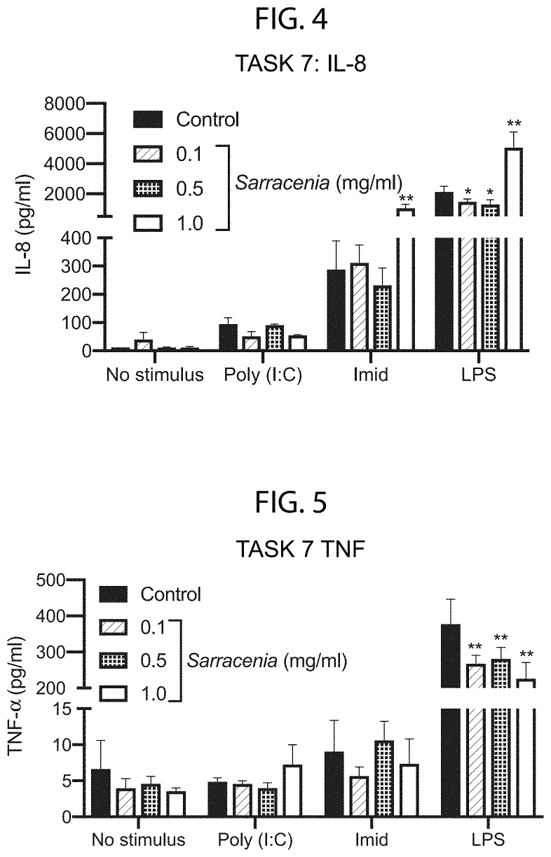

The Sarracenia extract was added at varying concentrations to wells containing the THP-1 cells and the TLR agonists. At the highest concentration of 1.0 mg/mL Sarracenia extract, there was an increase in Imid- and LPS-induced IL-8 release. With respect to TNF-α, the Sarracenia extract reduced the release of this cytokine at all concentrations tested when it was added to LPS-stimulated THP-1 cells but there were no significant changes observed in wells with other TLR agonists. Results are shown in FIGS. 4 and 5.

FIG. 4: Effect of formulations comprising Sarracenia extracts on IL-8 during TLR stimulation. Naïve THP-1 cells were exposed for 24 to poly(I:C) 100 μg/ml, imidazoquinoline (Imid) 1 μg/ml, or LPS 10 ng/ml in the presence or absence of three concentrations of the extract 0.1, 0.5 and 1.0 mg/ml). IL-8 release was measured by ELISA. Each value represents the mean±SD, n=3 samples. *p<0.05, and **p<0.01 vs stimulus Control (Dunnett' s multiple comparison test).

FIG. 5: Effect of formulations comprising Sarracenia extracts on TNF-α during TLR stimulation. Naïve THP-1 cells were exposed for 24 to poly(I:C) 100 μg/ml, imidazoquinoline (Imid) 1 μg/ml, or LPS 10 ng/ml in the presence or absence of three concentrations of the extract 0.1, 0.5 and 1.0 mg/ml). TNF-α release was measured by ELISA. Each value represents the mean±SD, n=3 samples. **p<0.01 vs LPS Control (Dunnett's multiple comparison test).

### Conclusions:

The Sarracenia extract had a modulatory effect on immune responses elicited by TLR agonists. The data suggest that Sarracenia extract minimally, in case of TLRs 4 and 7/8, modulates innate immune activation as assessed by pro-inflammatory cytokine release. A statistically significant increase in Imid (TLR 7/8 agonist) induced IL-8 release was observed at the highest concentration of Sarracenia tested. Imidazoquinoline compounds are used as topical immune response modifiers that lack direct antiviral effects; for example, Aldara (imiquimod) is used for treatment of external genital and perianal warts caused by human papilloma virus (HPV), for the treatment of superficial basal cell carcinoma, and for actinic keratosis (i.e. lesions on the skin caused by excessive sun exposure). IL-8 is a cytokine that is involved in recruitment of neutrophils and lymphocytes to sites of inflammation; therefore, the Sarracenia formulation may have immune enhancing effects when utilized in combination with specific TLR agonists.

### Example 4

### Assessment of Wound Healing by Formulations Comprising Sarracenia Extracts

### Objectives:

Wound healing is a complex process of tissue regeneration, among the fundamental processes in the reconstruction of damaged skin. In human wound healing, keratinocyte re-epithelialization is the most essential part, as the skin's primary objective is to quickly re-establish barrier function. To determine the wound healing capacity of the new Sarracenia extract, in vitro cell adhesion and wound scratch assays were performed as indicators for re-epithelialization in wound healing. These experiments used NCTC2544 cells, a commercially available skin epithelial-like cell line.

### Methods:

Cell Adhesion Assays: the effect on cell adhesion was evaluated by exposing the NCTC2544 cells to three non-cytotoxic concentrations (0.1, 0.5 and 1 mg/ml) of the extract during the adhesion phase. Adhesion was evaluated by colouring and fixing the cells with 0.1% crystal violet in 2% ethanol after 30 and 60 minutes of incubation. The result is expressed as OD.

Wound Scratch Healing Assays: confluent NCTC 2544 cells in 24-well plates were damaged by a scratch using a 20-μl pipette tips. After washing with PBS, cells were exposed to three non-cytotoxic concentrations (0.1, 0.5 and 1 mg/ml) of the Sarracenia extract or carrier as vehicle control, and re-epithelialization evaluated after 24 hours either by evaluating under the microscope the filling percentage documented using a digital camera. The result is expressed as % re-epithelialization versus the initial scratch area (t=0).

Each experiment was conducted in triplicate, and three independent experiments were conducted. Any statistically significant differences versus control cells was considered as relevant.

### Results:

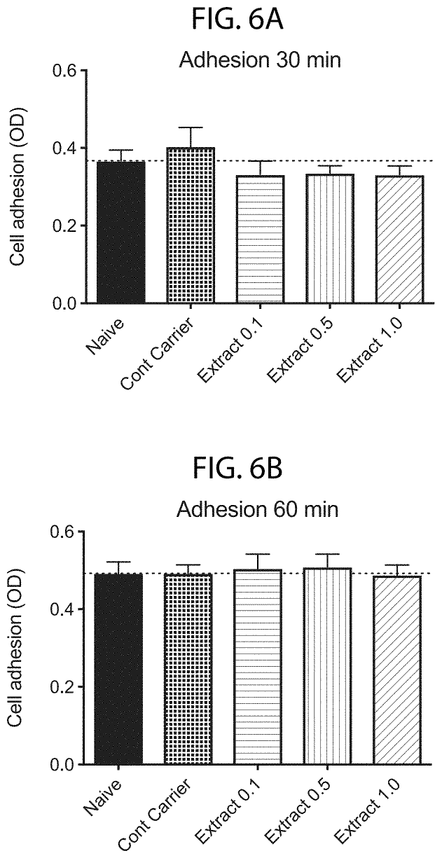

The effect on cell adhesion was evaluated by exposing the NCTC2544 cells to three non-cytotoxic concentrations (0.1, 0.5 and 1 mg/ml) of Sarracenia extract during the adhesion phase. The extract or the vehicle carrier did not affect cell adhesion at any concentration and time tested, showing values similar to untreated cells. Results are shown in FIGS. 6A and 6B.

FIGS. 6A and 6B: Effect of formulations comprising Sarracenia extracts on cell adhesion. Each column represents the mean±SD of the OD obtained in three independent experiments run in triplicate.

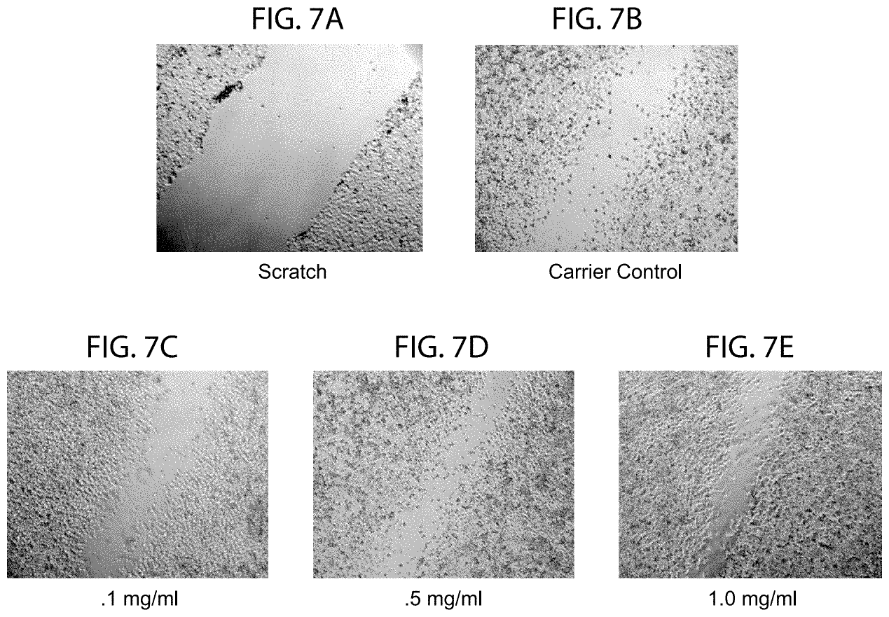

The effect of the new Sarracenia extract on wound scratch healing assay was observed. Confluent NCTC 2544 cells were damaged by a scratch using a 20-μl pipette tips, and exposed to three non-cytotoxic concentrations (0.1, 0.5 and 1 mg/ml) of the Sarracenia extract or the vehicle control. The extract or the carrier alone did not affect cell adhesion at any concentration and time tested, showing values similar to untreated cells (naïve). Results are shown in FIGS. 7A-7E. FIG. 7A is the scratch at time zero, and

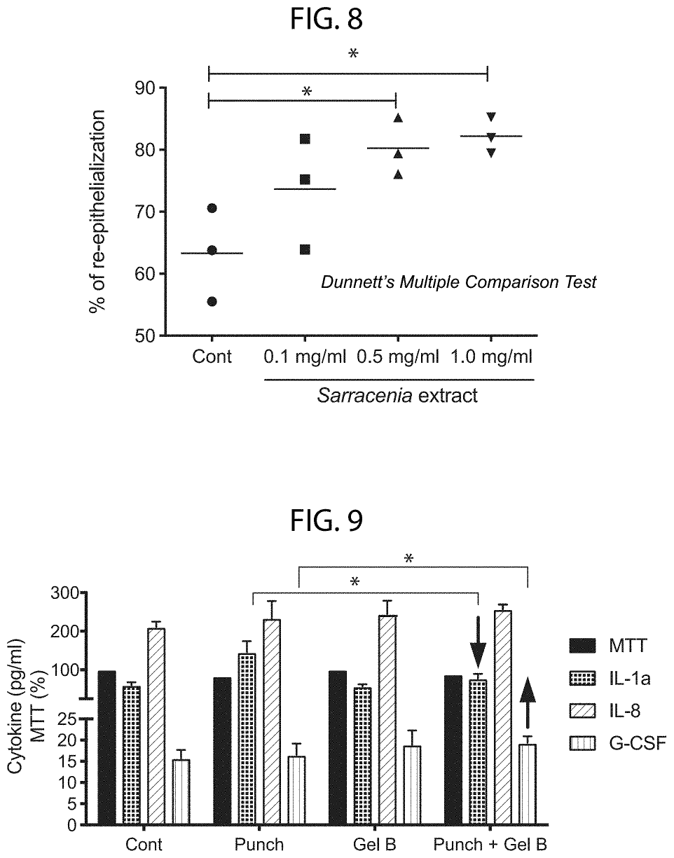

FIGS. 7A-7E and FIG. 8: Effect of formulations comprising Sarracenia extracts in an in vitro wound scratch healing assay. The photos are representative images of the scratch healing following 24 h of treatment with increasing concentrations of Sarracenia extract. The first photo, FIG. 7A is representative of the initial scratch (t=0), FIG. 7B shows the carrier control. FIGS. 7C, 7D, and 7E show the scratch healing of the following concentrations of Sarracenia: 0.1 mg/ml, 0.5 mg/ml, and 1.0 mg/ml, respectively. In FIG. 8, the % re-epithelialization versus scratched cells at t=0 is reported. Each dot represents the mean of the % of re-epithelialization obtained in independent experiments run in triplicate. Statistical analysis was performed with Dunnett' s multiple comparison test, with *p<0.05 vs carrier treated cells (Cont).

### Conclusions:

The Sarracenia extract induced a concentration-dependent and statistically significant increases in wound closure in a monolayer of human keratinocytes using the wound scratch assay compared to carrier treated cells, which is indicative of regeneration capacity (i.e. re-epithelialization) of the formulation. No changes in cell adhesion were observed.

### Example 5

### Evaluation of Formulations Comprising Sarracenia Extracts in a Three-Dimensional Model of Wound Healing

### Objectives:

A 3D organotypic skin model was used to assess the effects of the Sarracenia extract. As indicators of the ongoing wound healing process IL-1α, IL-6, IL-8 and granulocye-colony stimulating factor (G-CSF) release were evaluated [16]. In parallel, cell viability was assessed by the MTT assay.

### Methods:

Tissues were wounded by a circular 2-mm biopsy punch, 25 μl of the final product (Gel B) was then added or tissues were left untreated (control), and cultures incubated for 24 h by commercially available ELISA

### Results:

The analysis of cytokines revealed interesting results related to the effect of Sarracenia formulation on the release of IL-1α and G-CSF. IL-1α was reduced by the Sarracenia formulation, which would be expected to be beneficial for preventing formation of keloids or hypertrophic scars in wounded skin [17]. Studies in experimental animals showed that skin wounds in animals that are genetically deficient in IL-1 receptor had improved restoration of normal skin architecture marked by a reduced fibrotic response in wounds and accompanied by reduced inflammatory cell infiltration. Another finding was that the Sarracenia formulation caused a slight increase in G-CSF production at the wound site. G-CSF is a hematopoietic cytokine and potent stem cell mobilizing agent [18]. Scratch assay and tube formation experiments have also revealed that G-CSF facilitated migration ability and angiogenic potential of human umbilical vein endothelial cells (HUVEC) [19]. Results are shown in FIG. 9.

FIG. 9: Evaluation of formulations comprising Sarracenia extracts in a 3D model of wound healing. Skin tissues were wounded by a circular 2-mm biopsy punch, 25 μ1 of the Sarracenia formulation (Gel B) was then added or tissues left untreated for 24 h. As indicator of the ongoing wound healing process IL-1α, IL-6, IL-8 and G-CSF release were assessed. Each value represents the mean±SD, n=3. Statistical analysis was performed with Tukey's multiple comparison test with *p<0.05 vs Punch group.

### Conclusions:

These data indicate that the Sarracenia formulation has regenerative capabilities that promote re-epithelization of skin. In vivo, the Sarracenia formulation has benefits for modulating inflammation, reducing fibrosis, and mobilization of stem cells to sites of skin damage.

### REFERENCES

1. Hotti, H., et al., Metabolite profiling of the carnivorous pitcher plants Darlingtonia and Sarracenia. PLoS One, 2017. 12(2): p. e0171078.
2. Leduc, C., et al., Plants used by the Cree Nation of Eeyou Istchee (Quebec, Canada) for the treatment of diabetes: A novel approach in quantitative ethnobotany. J Ethnopharmacol, 2006. 105(1-2): p. 55-63.
3. Harris, C. S., et al., Characterizing the cytoprotective activity of Sarracenia purpurea L., a medicinal plant that inhibits glucotoxicity in PC12 cells. BMC Complement Altern Med, 2012. 12: p. 245.
4. Muhammad, A., et al., Antidiabetic compounds from Sarracenia purpurea used traditionally by the Eeyou Istchee Cree First Nation. J Nat Prod, 2012. 75(7): p. 1284-8.
5. Arndt, W., et al., In vitro characterization of a nineteenth-century therapy for smallpox.

PLoS One, 2012. 7(3): p. e32610.
6. Morrison, S. A., et al., Antimycobacterial triterpenes from the Canadian medicinal plant Sarracenia purpurea. J Ethnopharmacol, 2016. 188: p. 200-3.
7. Miles, D. H. and U. Kokpol, Tumor inhibitors II: constituents and antitumor activity of Sarracenia flava. J Pharm Sci, 1976. 65(2): p. 284-5.
8. Miles, D. H., et al., Tumor inhibitors. I. Preliminary investigation of antitumor activity of Sarracenia flava. J Pharm Sci, 1974. 63(4): p. 613-5.
9. Tarapore, R. S., et al., Specific targeting of Wnt/beta-catenin signaling in human melanoma cells by a dietary triterpene lupeol. Carcinogenesis, 2010. 31(10): p. 1844-53.
10. Csuk, R., et al., Synthesis, encapsulation and antitumor activity of new betulin derivatives. Arch Pharm (Weinheim), 2011. 344(1): p. 37-49.
11. Mullauer, F. B., J. H. Kessler, and J. P. Medema, Betulinic acid, a natural compound with potent anticancer effects. Anticancer Drugs, 2010. 21(3): p. 215-27.
12. Jia, H. X. and Y. L. He, Efficacy and safety of imiquimod 5% cream for basal cell carcinoma: a meta-analysis of randomized controlled trial. J Dermatolog Treat, 2019: p. 1-8
13. Nair, S., G. E. Archer, and T. F. Tedder, Isolation and generation of human dendritic cells. Curr Protoc Immunol, 2012. Chapter 7: p. Unit? 32.
14. Gilboa, E., DC-based cancer vaccines. J Clin Invest, 2007. 117(5): p. 1195-203.
15. Berges, C., et al., A cell line model for the differentiation of human dendritic cells. Biochem Biophys Res Commun, 2005. 333(3): p. 896-907.
16. Hanel, K. H., et al., Cytokines and the skin barrier. Int J Mol Sci, 2013. 14(4): p. 6720-45.
17. Thomay, A. A., et al., Disruption of interleukin-1 signaling improves the quality of wound healing. Am J Pathol, 2009. 174(6): p. 2129-36.
18. Patterson, A. M. and L. M. Pelus, G-CSF in stem cell mobilization: new insights, new questions. Ann Blood, 2017. 2.
19. Huang, H., et al., Granulocyte-Colony Stimulating Factor (G-CSF) Accelerates Wound Healing in Hemorrhagic Shock Rats by Enhancing Angiogenesis and Attenuating Apoptosis. Med Sci Monit, 2017. 23: p. 2644-2653.

1. A formulation comprising: a Sarracenia leucophylla extract selected from the group consisting of flower, leaf, seed, and stalk at 0.1-0.6% wt/wt, Aloe barbadensis leaf juice at 5-15% wt/wt, Lavandula angustifolia extract at 0.1-0.6% wt/wt, Melissa officinalis extract at 0.05-0.50% wt/wt, Melissa officinalis leaf extract at 0.01-0.1% wt/wt, Glycyrrhiza glabra root extract at 0.01-0.06% wt/wt, Hypericum perforatum extract at 0.01-0.06% wt/wt, Camellia sinensis leaf extract at 0.01-0.06% wt/wt, Acanthopanax senticosus root extract at 0.01-0.06% wt/wt, a herpes medication at 0.5-3% wt/wt and alcohol at 1-3% wt/wt.

2. The formulation of claim 1, wherein said Sarracenia leucophylla extract is at about 0.17% wt/wt, Aloe barbadensis leaf juice is at about 10% wt/wt, Lavandula angustifolia extract is at about 0.15% wt/wt, Melissa officinalis extract is at about 0.100% wt/wt, Melissa officinalis leaf extract is at about 0.06% wt/wt, Glycyrrhiza glabra root extract is at about 0.03% wt/wt, Hypericum perforatum extract is at about 0.03% wt/wt, Camellia sinensis leaf extract is at about 0.02% wt/wt, Acanthopanax senticosus root extract is at about 0.02% wt/wt, Alcohol at about 1.7% wt/wt, Allantoin at about 0.5% wt/wt, and Lidocaine HCL at about 0.5% wt/wt.

3. The formulation of claim 1, wherein said formulation is formulated in a gel for topical administration directly to a skin lesion.

4. The formulation of claim 1, wherein said formulation is used for the treatment of a condition selected from the group consisting of: a) viral infections; b) tumors; c) wound healing; d) pain; e) acne; f) rosacea; g) autoimmune skin disorders; h) contact dermatitis;
i) allergic dermatitis; j) irritant dermatitis; k) contact urticaria; and/or, 1) eczema.

5. A method for treating orofacial manifestations of HSV-1 comprising: a) identifying an individual afflicted with orofacial manifestations of HSV-1; b) administering a topical formulation comprising a Sarracenia extract to the orofacial manifestations of HSV-1 in a therapeutic amount.

6. The method of claim 5, wherein said formulation comprising a Sarracenia extract is formulated in a pharmaceutical composition selected from the group consisting of: a) gel; b) cream; c) lip balm; and a d) medicated spray.

7. The method of claim 5, wherein said formulation comprises Sarracenia leucophylla extract selected from the group consisting of flower, leaf, seed, and stalk at 0.1-0.6% wt/wt, Aloe barbadensis leaf juice at 5-15% wt/wt, Lavandula angustifolia extract at 0.1-0.6% wt/wt, Melissa officinalis extract at 0.05-0.50% wt/wt, Melissa officinalis leaf extract at 0.01-0.1% wt/wt, Glycyrrhiza glabra root extract at 0.01-0.06% wt/wt, Hypericum perforatum extract at 0.01-0.06% wt/wt, Camellia sinensis leaf extract at 0.01-0.06% wt/wt, Acanthopanax senticosus root extract at 0.01-0.06% wt/wt, a herpes medication at 0.5-3% wt/wt and alcohol at 1-3% wt/wt.

8. The method of claim 5, wherein the formulation is administered between 3-4 times daily.

9. A method for treating infections with HSV-2 comprising: a) identifying an individual afflicted with genital herpes; b) administering a topical formulation comprising a Sarracenia extract to the genital herpes in a therapeutic amount.

10. The method of claim 9, wherein said formulation comprising a Sarracenia extract is formulated in a pharmaceutical composition selected from the group consisting of: a) gel; b) cream; c) lip balm; and a d) medicated spray.

11. The method of claim 9, wherein said formulation comprises Sarracenia leucophylla extract selected from the group consisting of flower, leaf, seed, and stalk at 0.1-0.6% wt/wt, Aloe barbadensis leaf juice at 5-15% wt/wt, Lavandula angustifolia extract at 0.1-0.6% wt/wt, Melissa officinalis extract at 0.05-0.50% wt/wt, Melissa officinalis leaf extract at 0.01-0.1% wt/wt, Glycyrrhiza glabra root extract at 0.01-0.06% wt/wt, Hypericum perforatum extract at 0.01-0.06% wt/wt, Camellia sinensis leaf extract at 0.01-0.06% wt/wt, Acanthopanax senticosus root extract at 0.01-0.06% wt/wt, a herpes medication at 0.5-3% wt/wt and alcohol at 1-3% wt/wt.

12. The method of claim 9, wherein the formulation is administered between 3-4 times daily.

13. A method for inducing topical wound closure consisting of: a) identifying an individual with a topical wound; b) administration to said individual a formulation comprising lidocaine and an extract from Sarracenia leucophylla in a therapeutic amount.

14. The method of claim 13, wherein said formulation comprises: Sarracenia leucophylla extract at 0.1-0.6% wt/wt, Aloe barbadensis leaf juice at 5-15% wt/wt, Lavandula angustifolia extract at 0.1-0.6% wt/wt, Melissa officinalis extract at 0.05-0.50% wt/wt, Melissa officinalis leaf extract at 0.01-0.1% wt/wt, Glycyrrhiza glabra root extract at 0.01-0.06% wt/wt, Hypericum perforatum extract at 0.01-0.06% wt/wt, Camellia sinensis extract at 0.01-0.06% wt/wt, and Acanthopanax senticosus root extract at 0.01-0.06% wt/wt.

15. A method for inducing dendritic cell maturation comprising: a) harvesting of natural human monocytes; b) in vitro culturing said monocytes with recombinant human cytokines including GM-CSF to generate immature dendritic cells; and c) in vitro culturing of immature dendritic cells with a formulation comprising Sarracenia extracts.

16. The method of claim 15, wherein said formulation comprises Sarracenia leucophylla extracts selected from the group consisting of: flower, leaf, seed, and stalk extracts.

17. The method of claim 16, wherein said formulation is administered to said dendritic cell cultures at a concentration of 1,000 μg/mL.

18. The method of claim 16, wherein said formulation is administered to said dendritic cell cultures at a concentration of 500-2,500 μg/mL.

19. The formulation of claim 1, wherein the herpes medication is selected from the group consisting of Lidocaine HCL and Allantoin.
